SEQUENCE LISTING (6 sequences)

SEQ ID NO: 1 - protein, 116 aa, synthetic construct
EVQLLESGGGLVQPGGSLRLSCAASGFTFSNYAMSWVRQAPGKGLEWVSGISSSSGSTYYADSVKGRFTISRDNSKNTLYLQMNSLGAEDTAVYYCARRPTAFDYWGQGTLVTVSS

SEQ ID NO: 2 - protein, 110 aa, synthetic construct
QSVLTQPPSASGTPGQRVTISCTGSSSNIGSNSVTWYQQLPGTAPKLLIYADSKRPSGVPDRFSGSKSGTSASLAISGLRSEDEADYYCGSWDSSLNGYVFGGGTKLTVL

SEQ ID NO: 3 - protein, 471 aa, synthetic construct [sequence omitted]

SEQ ID NO: 4 - protein, 424 aa, synthetic construct
MSYKAAAGEDYKADCPPGNPAPTSNHGPDATEAEEDFVDPWTVQTSSAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQAYSYAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCVIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLLPLIAEHQARRKEVTDEIVKEFMTPRKLSFDFQ

SEQ ID NO: 5 - protein, 401 aa, synthetic construct
SNHGPDATEAEEDFVDPWTVQTSSAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQAYSYAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCVIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLLPLIAEHQARRKEVTDEIVKEFMTPRKLSFDFQ

SEQ ID NO: 6 - protein, 378 aa, synthetic construct
SAKGIDYDKLIVRFGSSKIDKELINRIERATGQRPHHFLRRGIFFSHRDMNQVLDAYENKKPFYLYTGRGPSSEAMHVGHLIPFIFTKWLQDVFNVPLVIQMTDDEKYLWKDLTLDQAYSYAVENAKDIIACGFDINKTFIFSDLDYMGMSSGFYKNVVKIQKHVTFNQVKGIFGFTDSDCIGKISFPAIQAAPSFSNSFPQIFRDRTDIQCLIPCVIDQDPYFRMTRDVAPRIGYPKPALLHSTFFPALQGAQTKMSASDPNSSIFLTDTAKQIKTKVNKHAFSGGRDTIEEHRQFGGNCDVDVSFMYLTFFLEDDDKLEQIRKDYTSGAMLTGELKKALIEVLLPLIAEHQARRKEVTDEIVKEFMTPRKLSFDFQ

(19) United States
(12) Patent Application Publication
(10) Pub. No.: US 2025/0171559 A1
(43) Pub. Date: May 29, 2025

(54) METHOD OF TREATING CANCER USING WRS INHIBITORS

(71) Applicants: KOREA RESEARCH INSTITUTE OF BIOSCIENCE AND BIOTECHNOLOGY, Daejeon (KR); EWHA UNIVERSITY - INDUSTRY COLLABORATION FOUNDATION, Seoul (KR); MEDICINAL BIOCONVERGENCE RESEARCH CENTER, Incheon (KR)

(72) Inventors: Tae-Don KIM, Daejeon (KR); Young Ha AHN, Daejeon (KR); Sunghoon KIM, Suwon-si (KR); Hyunbo SHIM, Seoul (KR); Se-Chan OH, Daejeon (KR)

(73) Assignees: KOREA RESEARCH INSTITUTE OF BIOSCIENCE AND BIOTECHNOLOGY, Daejeon (KR); EWHA UNIVERSITY - INDUSTRY COLLABORATION FOUNDATION, Seoul (KR); MEDICINAL BIOCONVERGENCE RESEARCH CENTER, Incheon (KR)

(21) Appl. No.: 18/962,123

(22) Filed: Nov. 27, 2024

(30) Foreign Application Priority Data
Nov. 28, 2023 (KR) 10-2023-0168343

(51) Int. Cl. C07K 16/40; A61K 31/713; A61K 45/06; A61P 35/00; G01N 33/50; G01N 33/68

(52) U.S. Cl. CPC C07K 16/40; A61K 31/713; A61K 45/06; A61P 35/00; G01N 33/5011; G01N 33/6893; C07K 2317/51; C07K 2317/515; C07K 2317/56; G01N 2500/10

(57) ABSTRACT

The present disclosure relates to a method of treating cancer using a tryptophanyl-tRNA synthetase (WRS) inhibitor. The WRS is secreted by cancer cells and induces or promotes differentiation of myeloid-derived suppressor cells (MDSCs), which may inhibit anticancer immunity of immune cells, and therefore, the WRS inhibitor may effectively inhibit proliferation of cancer cells by inhibiting expression or activity of the WRS and inhibiting differentiation of MDSCs. Accordingly, the present disclosure may be used as an effective method of ameliorating or treating cancer and a method of enhancing or promoting anticancer efficacy of an anticancer drug, and further, may be usefully utilized as a method of screening a substance that inhibits the expression or activity of WRS as an anticancer drug.

### CROSS-REFERENCE TO RELATED APPLICATION

This application is based on and claims the benefit of priority to Korean Patent Application No. 10-2023-0168343, filed on Nov. 28, 2023 in the Korean Intellectual Property Office, the disclosure of which is incorporated herein in its entirety by reference.

### TECHNICAL FIELD

The present disclosure relates to a method of treating cancer using a tryptophanyl-tRNA synthetase (WRS) inhibitor.

### BACKGROUND

Unlike normal cells that are capable of performing regular and temperate proliferation and inhibition according to needs of an individual, cancer is a cell mass composed of undifferentiated cells that ignore necessary conditions in tissue and undergo unlimited proliferation, and is also called tumor. Cancer cells with the unlimited proliferation infiltrate surrounding tissue, and in more serious cases, the cancer cells metastasize to other organs of the body, which may cause severe pain and eventually lead to death. Cancer has a high mortality rate worldwide, and in particular, the incidence of various cancers such as colon cancer, breast cancer, and prostate cancer is continuously increasing due to aging population, a widespread consumption of high-fat diets, a rapid increase in environmental pollutants, and an increase in alcohol consumption. In this situation, there is an urgent need to create an anticancer substance that may contribute to enhancement of human health, improvement of quality of healthy life, and promotion of human health care by enabling early prevention and treatment of cancer.

Meanwhile, cancer may be largely classified into a blood cancer and a solid cancer, and may develop in almost all parts of the body such as stomach, pancreas, breast, mouth, liver, uterus, esophagus, and skin. As a cancer treatment method, recently, a few targeted agents such as Gleevec and Herceptin are used for treating certain but until now, surgery, radiation therapy, and anticancer drug treatment using chemotherapeutic drugs that suppresses cell proliferation are mainly performed. However, since the chemotherapeutic agents are not targeted agents, the biggest problems with the existing chemotherapeutic agents are side effects due to cytotoxicity and drug resistance, which are the main factors in which the treatment eventually fails despite the initial successful response by the anticancer drug. Therefore, in order to overcome the limitations of such chemotherapeutic agents, it is necessary to continuously develop targeted agents having clear anticancer action mechanisms.

### SUMMARY

An aspect of the present disclosure provides a method of ameliorating or treating cancer.

Another aspect of the present disclosure provides a method of enhancing or promoting anticancer efficacy of an anticancer drug.

Still another aspect of the present disclosure provides a novel method of screening an anticancer drug.

Still another aspect of the present disclosure provides an antibody that specifically binds to tryptophanyl-tRNA synthetase (WRS) or an antigen-binding fragment thereof.

According to an exemplary embodiment of the present disclosure, a method of ameliorating or treating cancer includes administering a tryptophanyl-tRNA synthetase (WRS) inhibitor to a subject in need thereof.

According to another exemplary embodiment of the present disclosure, a method of enhancing or promoting anticancer efficacy anticancer drug includes administering a tryptophanyl-tRNA synthetase (WRS) inhibitor to a subject in need thereof.

According to still another exemplary embodiment of the present disclosure, a method of screening an anticancer drug includes: treating a cell expressing tryptophanyl-tRNA synthetase (WRS) with a candidate substance; measuring an expression level or activity level of the WRS in the cell treated with the candidate substance; and determining the candidate substance as an anticancer drug when the expression level or activity level of the WRS decreases compared to a control group not treated with the candidate substance.

According to still another exemplary embodiment of the present disclosure, there is provided an antibody that specifically binds to tryptophanyl-tRNA synthetase (WRS) or an antigen-binding fragment thereof, the antibody or the antigen-binding fragment thereof containing a heavy chain variable region containing an amino acid sequence of SEQ ID NO: 1 and a light chain variable region containing an amino acid sequence of SEQ ID NO: 2.

### BRIEF DESCRIPTION OF THE DRAWINGS

The above and other objects, features and advantages of the present disclosure will be more apparent from the following detailed description taken in conjunction with the accompanying drawings.

FIG. 1 illustrates the results of measuring WRS expression levels after co-culturing human monocytes with normal cell line or a cancer cell line.

FIG. 2 illustrates the results of measuring WRS expression levels in a whole cell lysate (WCL) and a supernatant (SUP) after co-culturing human monocytes with ovarian cancer cell line SKOV3.

FIG. 3 illustrates the results of measuring WRS expression levels after co-culturing human immune cells with ovarian cancer cell line SKOV3.

FIG. 4A illustrates the results of short-term WRS expression in a peritoneal exudate cell (PEC) and a peritoneal lavage (PL) measured using Western blotting and ELISA after intraperitoneal injection of cancer cell line YAC-1 into a mouse.

FIG. 4B illustrates the results of long-term WRS expression in a peritoneal exudate cell (PEC) and a peritoneal lavage (PL) measured using Western blotting and RT-qPCR after intraperitoneal injection of cancer cell line YAC-1 into a mouse.

FIG. 5 illustrates the results of measuring a level of MDSC differentiation after intraperitoneal injection of cancer cell line YAC-1 into a mouse.

FIG. 6 illustrates the results of measuring a level of MDSC differentiation after intraperitoneal injection of WRS or fragments thereof into a mouse.

FIG. 7 illustrates the results of measuring a level of MDSC differentiation by flow cytometry after intraperitoneal injection of cancer cell line YAC-1 and full-length WRS or mini-WRS together into a mouse.

FIG. 8A illustrates the results of measuring a level of MDSC differentiation after treating human umbilical cord blood CD34⁺ hematopoietic stem cells (HSCs) with hGM-CSF to differentiate the HSCs into MDSCs, and then performing treatment with WRS or fragments thereof.

FIG. 8B illustrates the results of measuring a level of MDSC differentiation after treating mouse bone marrow hematopoietic stem cells with mGM-CSF to differentiate the stem cells into MDSCs, and then performing treatment with WRS or fragments thereof.

FIG. 9 illustrates the results of measuring a tumor volume and a level of MDSC differentiation after forming a solid cancer by implanting cancer cell line 4T1 subcutaneously in a mouse and injecting mini-WRS into a cancer nodule.

FIG. 10 illustrates the results of measuring a level of T-cell differentiation by flow cytometry after intraperitoneal injection of cancer cell line YAC-1 and full-length WRS or mini-WRS together into a mouse.

FIG. 11 illustrates the results of measuring a level of T cell differentiation and proliferation after co-culturing an FBL cell line expressing gag as an antigen and a splenocyte from a TCR-gag transgenic mouse to proliferate antigen-specific T cells, and performing treatment with full-length WRS or mini-WRS.

FIG. 12 illustrates the results of measuring binding affinity of WRS and fragments thereof to an LILRB receptor and binding affinity to mini-WRS cells after inhibiting the expression of LILRB1 and LILRB2 in monocyte cell line U937.

FIG. 13A illustrates a schematic diagram of antibodies to WRS and fragments thereof.

FIG. 13B illustrates the results of Western blotting analysis of binding affinity of antibodies to WRS and fragments thereof.

FIG. 13C illustrates the results of SPR analysis of binding affinity of antibodies to WRS and fragments thereof.

FIG. 14 illustrates the results of measuring a tumor volume after forming a solid cancer by implanting cancer cell line 4T1 subcutaneously in a mouse and injecting a WRS antibody into a cancer nodule.

FIG. 15 illustrates the results of measuring a level of MDSC differentiation in the spleen, bone marrow, and blood after treatment with WRS, fragments thereof, or antibodies thereto.

FIG. 16 illustrates a schematic diagram of a mechanism by which MDSC differentiation may be inhibited through WRS inhibition, thereby activating immune cells to inhibit tumor cells.

### DETAILED DESCRIPTION

Hereinafter, the present disclosure will be described in detail.

### 1. Method of Ameliorating or Treating Cancer

An aspect of the present disclosure provides a method of ameliorating or treating cancer.

The method of ameliorating or treating cancer of the present disclosure includes administering a tryptophanyl-tRNA synthetase (WRS) inhibitor to a subject in need thereof.

The “WRS”, which is an essential enzyme that binds tryptophan to tRNA through aminoacylation during a protein translation process, is expressed by IFNg and secreted from immune cells in response to bacterial or viral infection, and is known to play a physiopathological role in various diseases including sepsis, autoimmune diseases, and brain diseases.

Meanwhile, eukaryotic WRS is genetically conserved with a Rossmann fold (RF) domain, an anticodon binding domain (ABD), and a eukaryotic-specific extension (ESE) domain, and full-length WRS (SEQ ID NO: 3) consists of a total of 471 amino acids and contains a vertebrate-specific extension (VES) domain known as a WHEP domain. The full-length WRS may be spliced or truncated by a protease during a transcription process to form fragments such as mini-WRS, T1-WRS, and T2-WRS. Based on the full-length WRS, the mini-WRS (SEQ ID NO: 4) consists of a total of 424 amino acids from 48 to 471, the T1-WRS (SEQ ID NO: 5) consists of a total of 401 amino acids from 71 to 471, and the T2-WRS (SEQ ID NO: 6) consists of a total of 378 amino acids from 94 to 471.

In the present disclosure, the WRS may include not only full-length WRS, but also fragments generated by splicing or truncating the full-length WRS by a protease during the transcription process. The WRS fragments may be specifically mini-WRS, T1-WRS, and T2-WRS, and more specifically, mini-WRS and T1-WRS.

In the present disclosure, the WRS inhibitor is used as a general term for all agents that reduce the expression or activity of WRS, and may specifically include an expression inhibitor that reduces the expression of WRS at a transcription, mRNA, or translation level, either by affecting a reduction in expression of WRS or by acting directly on WRS or indirectly on a ligand thereof, and an active inhibitor that reduces the activity of WRS by directly binding to a WRS protein or a ligand that binds to WRS and thereby inhibiting the activity thereof. In addition, the inhibition of the WRS may be achieved by partially or entirely mutating, substituting, or deleting the WRS gene by a genome editing system (for example, a CRISPR/Cas9 system), or by inserting one or more bases into the WRS gene.

For example, the WRS expression inhibitor may complementarily bind to the WRS gene, and specifically, the WRS expression inhibitor may be an antisense oligonucleotide, siRNA, shRNA, miRNA, or the like.

The “antisense oligonucleotide” is DNA, RNA, or a derivative thereof containing a nucleic acid sequence complementary to a sequence of specific mRNA, and the antisense oligonucleotide binds to a complementary sequence in mRNA and acts to inhibit the translation of mRNA into a protein. A sequence of the antisense oligonucleotide refers to a DNA or RNA sequence that is complementary to the WRS mRNA and may bind to the mRNA. The sequence may inhibit the essential activity for translation, translocation into the cytoplasm, maturation, or all other overall biological functions of the WRS mRNA. The antisense oligonucleotide may be synthesized in vitro in a conventional method to be administered in vivo, or the antisense oligonucleotide may be synthesized in vivo. One example of synthesizing the antisense oligonucleotide in vitro is to use RNA polymerase I. One example of synthesizing the antisense RNA in vivo is to allow the antisense RNA to be transcribed by using a vector having the origin of a multicloning site (MCS) in an opposite direction. It is preferable that the antisense RNA is not translated into a peptide sequence so that a translation stop codon is present in the sequence. A design of the antisense oligonucleotide that may be used in the present disclosure may be made according to a method known in the art with reference to the base sequence of WRS.

The siRNA and shRNA are nucleic acid molecules capable of mediating RNA interference (RNAi) or genetic silencing, and are used as effective gene knock down methods or gene therapy methods because they may inhibit the expression of a target gene. The shRNA has a hairpin structure formed by binding between Complementary sequences in a single-stranded oligonucleotide, and the shRNA in vivo is cleaved by a dicer to form siRNA, which is a double-stranded oligonucleotide of a small RNA fragment of 21 to 25 nucleotides in size, and may specifically bind to mRNA with a complementary sequence to inhibit the expression. Therefore, which means of shRNA and siRNA to use may be determined by those skilled in the art, and when the mRNA sequences targeted thereby are identical, similar expression reduction effects may b expected. For the purpose of the present disclosure, the siRNA and shRNA may specifically act on WRS to truncate WRS mRNA molecules and induce RNA interference, thereby inhibiting the WRS. The siRNA may be chemically or enzymatically synthesized. A method of preparing siRNA is not particularly limited, and methods known in the art may be used. For example, the method of preparing siRNA include a method of directly chemically synthesizing siRNA, a method of synthesizing siRNA using in vitro transcription, a method of truncating long double-stranded RNA synthesized by in vitro transcription using an enzyme, an expression method through intracellular delivery of an shRNA expression plasmid or viral vector, an expression method through intracellular delivery of a polymerase chain reaction (PCR)-induced siRNA expression cassette, and the like, but is not limited thereto.

The miRNA refers to a small non-coding RNA molecule of about 15 to 50 nucleotides in length, and preferably 17 to 23 nucleotides in length, which regulates the expression of a target gene after transcription. Biogenesis of the miRNA may be a multi-step process occurring in the cell nucleus and cytoplasm. Mature miRNA may be incorporated into an RNA-induced silencing complex to bind to the 3′-end untranslated region (UTR) of mRNA, which may induce mRNA degradation or translational inhibition. The miRNA may be processed from a hairpin precursor (pre-miRNA) of about 70 or more nucleotides derived from a primary transcript (pri-miRNA) through sequential truncation by RNAse III enzyme within the cell.

In the present disclosure, in order to increase the efficiency of delivering a nucleic acid material such as an antisense oligonucleotide, siRNA, shRNA, or miRNA into a cell, a safe and efficient delivery system may be used. For example, the antisense oligonucleotide, siRNA, shRNA, or miRNA of the present disclosure may be contained in a composition together with a nucleic acid delivery system. In the present disclosure, the nucleic acid delivery system may be broadly divided into a viral vector and a nonviral vector. The most widely used system is a viral vector because the delivery efficiency is high and the time of duration is long. Among various viral vectors, a retroviral vector, an adenoviral vector, an adeno-associated viral vector, and the like are mainly used. The nonviral vector has the advantages of low toxicity and immune response, may be administered repeatedly, is easy to form a complex with a ribonucleic acid, and is easy to mass produce. In addition, a specific ligand at a disease cell or a tissue site is conjugated to a nonviral vector, which enables a long-term cell selective nucleic acid delivery. As the nonviral vector, a variety of formulations, such as micelles, emulsions, and nanoparticles, including liposomes and cationic polymers, may be used. The nucleic acid delivery system may significantly enhance delivery efficiency of a desired nucleic acid in animal cells and the nucleic acid may be delivered even to any animal cells in accordance with an intended use of the nucleic acid to be delivered.

In addition, the WRS activity inhibitor may specifically bind to the WRS protein, and for example, the WRS activity inhibitor may be a small molecule compound, an aptamer, an antibody, or the like.

The small molecule compound is an organic compound having a molecular weight of 1,000 Da or less, and refers to a molecule that often acts as a regulator in a biological process and binds to a biopolymer such as a protein or a nucleic acid to regulate the function of the biopolymer. The small molecule compound may inhibit protein function or interfere with protein-protein interactions, and may be synthesized artificially. In the present disclosure, the small molecule compound may bind to WRS or a ligand protein of WRS and may inhibit the activity of WRS.

The aptamer is a single-stranded oligonucleotide of 20 to 60 nucleotides in size, and refers to a nucleic acid molecule having binding activity for a specific target molecule. The aptamer has diverse tertiary structures according to a sequence, may have high affinity to a specific substance like in an antigen-antibody reaction, and may bind to a specific target molecule and inhibit the activity of the specific target molecule. The aptamer may be RNA, DNA, a modified nucleic acid, or a mixture thereof, and may be in a linear or cyclic form. In the present disclosure, the aptamer may bind to WRS and inhibit the activity of WRS, and may be prepared by those skilled in the art from the sequence of WRS by a method known in the art.

The antibody refers to a substance that reacts to an antigen, which is a foreign substance, when the antigen invades the body, while circulating the blood or lymph in the immune system of the body, and is a globulin-based protein formed in lymphatic tissue and is also called immunoglobulin. The antibody is a protein produced by B cells and released into a body fluid and specifically binds to an antigen, one antibody molecule contains two heavy chains and two light chains, and each of the heavy chain and the light chain has a variable region at its N-terminal end. Each variable region consists of three complementarity determining regions (CDRs) and four framework regions (FRs), and the complementarity determining regions determine an antigen-binding specificity of an antibody and exist as relatively short peptide sequences that are maintained by a framework region of the variable region.

In the present disclosure, the antibody may specifically and directly bind to the WRS protein and efficiently inhibit the activity of the WRS protein. The antibody that specifically binds to the WRS may be prepared by a method known to those skilled in the art, and may be prepared, for example, by injecting the WRS protein, which is an immunogen, into an external host. The external host may include mammals such as a mouse, a rat, sheep, and a rabbit, and the immunogen may be injected intramuscularly, intraperitoneally, or subcutaneously, and generally may be administered together with an adjuvant to enhance antigenicity. Blood may be collected from the external host at regular intervals and serum exhibiting specificity to the antigen may be collected to isolate an antibody therefrom.

In addition, in the present disclosure, the antibody may specifically and directly bind to full-length WRS as well as fragments generated by splicing or protease truncation of the full-length WRS during a transcription process. The WRS fragments may be specifically mini-WRS, T1-WRS, and T2-WRS, and more specifically, mini-WRS and T1-WRS. In other words, in the present disclosure, the antibody may bind to mini-WRS or T1-WRS and effectively inhibit the activity thereof, thereby inhibiting the differentiation of and MDSCs effectively inhibiting the proliferation of cancer cells. In the present disclosure, it was confirmed that various antibodies binding to mini-WRS or T1-WRS may effectively inhibit the proliferation of cancer cells by inhibiting the differentiation of MDSCs.

As an example, in the present disclosure, the antibody to WRS may contain a heavy chain variable region containing an amino acid sequence of SEQ ID NO: 1 and a light chain variable region containing an amino acid sequence of SEQ ID NO: 2, but is not limited thereto. The antibody containing the heavy chain variable region containing the amino acid sequence of SEQ ID NO: 1 and the light chain variable region containing the amino acid sequence of SEQ ID NO: 2 not only may inhibit the activity of WRS, but may also effectively inhibit the activity of mini-WRS fragments in particular, thereby significantly inhibiting the proliferation of cancer cells.

In addition, the amino acid sequence of SEQ ID NO: 1 or SEQ ID NO: 2 may contain a polypeptide containing an amino acid sequence substantially identical to the amino acid sequence of SEQ ID NO: 1 or SEQ ID NO: 2, and a variant or active fragment thereof. The polypeptide containing substantially the same amino acid sequence refers to a polypeptide containing an amino acid sequence having a sequence homology of about 70% or more, about 75% or more, about 80% or more, about 85% or more, about 90% or more, about 92% or more, about 95% or more, about 97% or more, about 98% or more, or about 99% or more to the amino acid sequence of SEQ ID NO: 1 or SEQ ID NO: 2, respectively.

Meanwhile, in the present disclosure, the WRS inhibitor may inhibit differentiation of myeloid-derived suppressor cells (MDSCs).

The myeloid-derived suppressor cells (MDSCs) are known to promote the proliferation of cancer cells by inducing differentiation by cancer and inhibiting anticancer immunity of immune cells.

In the present disclosure, the WRS inhibitor may inhibit the proliferation of cancer cells by inhibiting the differentiation of MDSCs, and thus may be usefully utilized in a method of preventing or treating cancer and a method of enhancing or promoting anticancer efficacy of an anticancer drug.

In a specific exemplary embodiment of the present disclosure, WRS and fragments thereof promote the differentiation of MDSCs and the growth of cancer cells, while target antibodies to the WRS or fragments thereof as WRS inhibitors effectively inhibit the differentiation of MDSCs and the growth of cancer cells. Accordingly, it was confirmed that the WRS inhibitor of the present disclosure may be usefully utilized in a method of preventing or treating cancer and a method of enhancing or promoting anticancer efficacy of an anticancer drug.

The “cancer” refers to any condition in which a problem medically occurs to regulatory functions of cells for normal division, differentiation, or death, cells abnormally proliferate excessively and invade surrounding tissue or organs to form a mass, and an existing structure is destroyed or transformed.

In the present disclosure, the cancer may include all cancers and a specific disease name is not particularly limited as long as it falls within the medical category of cancer. Specifically, the cancer may be one or more selected from the group consisting of stomach cancer, colon cancer, colorectal cancer, rectal cancer, esophageal cancer, pancreatic cancer, liver cancer, cervical cancer, breast cancer, ovarian cancer, head and neck cancer, bone cancer, leukemia, carcinoid, prostate cancer, lung cancer, bladder cancer, endometrial cancer, melanoma, kidney cancer, testicular cancer, glioma, thyroid cancer, skin cancer, and lymphoma, and more specifically, the cancer may be one or more selected from the group consisting of lung cancer, breast cancer, and ovarian cancer. In addition, in the present disclosure, differentiation of MDSCs may be promoted by the cancer, and hyperdifferentiation of the MDSCs may further promote cancer proliferation.

Meanwhile, in the present disclosure, the “prevention” refers to any action that inhibits or delays the onset of cancer by administering the WRS inhibitor of the present disclosure, and the “treatment” refers to any action by which symptoms of cancer are improved or beneficially changed by administration of the WRS inhibitor of the present disclosure.

In addition, in the present disclosure, the “administration” refers to physical introduction of the WRS inhibitor of the present disclosure to a subject using any of various methods and delivery systems known to those skilled in the art. In the present disclosure, the “subject” refers to a subject requiring prevention or treatment of cancer, and specifically, refers to mammals such as a human or a primate, a mouse, a rat, a dog, a cat, a horse, a pig, a rabbit, or a cow.

The WRS inhibitor of the present disclosure may be provided in the form of a pharmaceutical composition, and the pharmaceutical composition may contain an active ingredient alone or may contain one or more pharmaceutically acceptable carriers, excipients, or diluents.

The “pharmaceutically acceptable” means that a substance does not inhibit activity of an active ingredient and does not have toxicity beyond what an application (prescription) target is adaptable to, and the “carrier” is defined as a compound that facilitates addition of a compound into cells or tissue.

Specifically, the carrier may be, for example, a colloidal suspension, a powder, a saline, a lipid, a liposome, a microsphere, or a nanospheric particle. These carriers may form or be related to a complex with a carrying means and may be carried in vivo by using a carrying system known in the art, such as a lipid, a liposome, a microparticle, gold, a nanoparticle, a polymer, a condensation reactant, polysaccharides, a polyamino acid, a dendrimer, saponin, an adsorption enhancing substance, or a fatty acid.

When the pharmaceutical composition of the present disclosure is formulated, the pharmaceutical composition may be prepared using a diluent or an excipient such as a commonly used lubricant, sweetener, flavoring agent, emulsifier, suspending agent, preservative, filler, extender, binder, wetting agent, disintegrating agent, or surfactant. A solid formulation for oral administration may include a tablet, a pill, a powder, a granule, a capsule, and the like. Such a solid formulation may be prepared by mixing the pharmaceutical composition with at least one excipient, for example, starch, calcium carbonate, sucrose, lactose, gelatin, or the like. In addition, in addition to the simple excipient, a lubricant such as magnesium stearate or talc may be used, A liquid formulation for oral administration includes a suspending agent, an oral liquid, an emulsion, a syrup, and the like, and various excipients such as a wetting agent, a sweetener, a fragrance, and a preservative, in addition to water and liquid paraffin, which are commonly used simple diluents, may be included. A formulation for parenteral administration includes a sterilized aqueous solution, a non-aqueous solvent, a suspending agent, an emulsion, a freeze-dried formulation, and a suppository. As the non-aqueous solvent and the suspending agent, propylene glycol, polyethylene glycol, vegetable oil such as olive oil, an injectable ester such as ethyl oleate, and the like may be used. As a base for the suppository, Witepsol, Macrogol, Tween 61, cacao butter, laurin butter, glycerogelatin, and the like may be used, and when the pharmaceutical composition of the present disclosure is prepared in the form of eye drops, a known diluent, excipient, or the like may be used.

Meanwhile, the pharmaceutical composition of the present disclosure may be appropriately administered to a subject according to a general method, administration route, and dosage used in the art depending on the purpose or need. Examples of the administration route include oral administration, parenteral administration, subcutaneous administration, intraperitoneal administration, intrapulmonary administration, and intranasal administration, and examples of parenteral injection include intramuscular administration, intravenous administration, intraarterial administration, intraperitoneal administration, and subcutaneous administration. In addition, an appropriate dosage and frequency of administration may be selected according to a method known in the art, and the amount of the pharmaceutical composition of the present disclosure actually administered and frequency of administration may be appropriately determined by various factors such as the type of symptom to be treated, administration route, gender, health condition, diet, age and weight of a subject, and severity of a disease.

The pharmaceutical composition of the present disclosure is administered in a pharmaceutically effective amount. The “pharmaceutically effective amount” refers to an amount sufficient to suppress or alleviate a disease at a reasonable rate applicable to medical use. An effective dose level may be determined according to factors including subject type, severity, age, gender, activity of a drug, sensitivity to a drug, an administration time, an administration route, a dissolution rate, a duration of treatment, and drugs used simultaneously, and other factors well-known in the medical field. The pharmaceutical composition of the present disclosure may be administered as an individual therapeutic agent or in combination with another therapeutic agent, and may be administered sequentially or simultaneously with a conventional therapeutic agent. In addition, the pharmaceutical composition may be administered once or multiple times. Considering all the factors, it is important to administer the pharmaceutical composition in an amount capable of obtaining a maximum effect with a minimal amount without side-effects, and the amount of the pharmaceutical composition to be administered may be easily determined by those skilled in the art. For example, the pharmaceutically effective amount may be 0.5 to 1, 000 mg/day/kg, and specifically, may be 0.5 to 500 mg/day/kg.

In addition, another aspect of the present disclosure provides a method of enhancing or promoting anticancer efficacy of an anticancer drug.

The method of enhancing or promoting anticancer efficacy of an anticancer drug of the present disclosure includes administering a tryptophanyl-tRNA synthetase (WRS) inhibitor to a subject in need thereof.

The WRS inhibitor of the present disclosure may function as an anticancer adjuvant and may thus be utilized in a method of enhancing or promoting anticancer efficacy of an anticancer drug.

In the present disclosure, the “anticancer adjuvant” refers to a formulation that may improve, enhance, or increase anticancer efficacy of an anticancer drug. The anticancer adjuvant may be administered simultaneously with, separately from, or sequentially in combination with an anticancer drug to enhance the anticancer efficacy of the anticancer drug, for example, the effect of inhibiting cancer cell growth or the effect of inhibiting cancer metastasis, to enhance the anticancer efficacy of the anticancer drug on anticancer drug-resistant cancer, or to suppress or improve the side effects of the anticancer drug.

In the present disclosure, the anticancer drug may include all a chemotherapy drug, a cancer vaccine, or a cancer immunotherapy drug, and in particular may be a cancer immunotherapy drug,

The cancer immunotherapy drug refers to an anticancer drug that stimulates the immune system and induces an immune response in which immune cells attack cancer cells, and may be an immune cell therapy agent, an immune checkpoint inhibitor, or an immune virus therapy agent. The immune cell therapy agent may be any one of a natural killer cell, a dendritic cell, a chimeric antigen receptor-expressing T cell, a T cell receptor T cell, a cytotoxic T lymphocyte, a tumor-infiltrating lymphocyte, and a chimeric antigen receptor-expressing natural killer cell.

In the method of enhancing or promoting anticancer efficacy of an anticancer drug of the present disclosure, the WRS inhibitor may be administered alone before or after administration of the anticancer drug, or may be administered together with the anticancer drug as an adjuvant for cancer treatment. When the anticancer adjuvant of the present disclosure is administered together with the anticancer drug, the anticancer adjuvant may be administered together with the anticancer drug in an appropriate ratio depending on conditions of a patient, a dosage of the anticancer drug, an administration period of the anticancer drug, and the like, and specifically, the anticancer adjuvant may be administered in an amount of 0.01 to 10 times the total weight of the anticancer drug.

In addition, still another aspect of the present disclosure provides an antibody that specifically binds to tryptophanyl-tRNA synthetase (WRS) or an antigen-binding fragment thereof.

The antibody or the antigen-binding fragment thereof may contain a heavy chain variable region containing an amino acid sequence of SEQ ID NO: 1 and a light chain variable region containing an amino acid sequence of SEQ ID NO: 2.

The amino acid sequence of SEQ ID NO: 1 or SEQ ID NO: 2 may contain a polypeptide containing an amino acid sequence substantially identical to the amino acid sequence of SEQ ID NO: 1 or SEQ ID NO: 2, and a variant or active fragment thereof. The polypeptide containing substantially the same amino acid sequence refers to a polypeptide containing an amino acid sequence having a sequence homology of about 70% or more, about 75% or more, about 80% or more, about 85% or more, about 90% or more, about 92% or more, about 95% or more, about 97% or more, about 98% or more, or about 99% or more to the amino acid sequence of SEQ ID NO: 1 or SEQ ID NO: 2.

The “antigen-binding fragment” refers to a portion of an intact antibody, in particular, any polypeptide or glycoprotein containing an antigen-binding site or variable region of an intact antibody, The antigen-binding fragment may be produced by recombinant DNA techniques or enzymatic or chemical degradation of an intact antibody, and examples of the antigen-binding fragment include, but are not limited to, Fv, Fab, F(ab′)2, Fab′, dsFv, (dsFv)2, ScFv, Sc(Fv)2, diabodies, and bispecific and multispecific antibodies formed from these antigen-binding fragments.

The Fab has a structure containing a variable region of each of a heavy chain and a light chain, a constant region of a light chain, and a first constant region of a heavy chain (CH1 domain), and has one antigen-binding site. The Fab′ differs from the Fab in that it has a hinge region containing one or more cysteine residues at the C-terminus of the heavy chain CH1 domain, The F(ab′)2 is produced when cysteine residues in the hinge region of Fab′ form a disulfide bond. The Fv refers to a minimal antibody fragment containing only a heavy chain variable region and a light chain variable region. The two-chain Fv is a fragment in which a heavy chain variable region and a light chain variable region are linked by a non-covalent bond, and single-chain Fv is a fragment in which a heavy chain variable region and a light chain variable region are generally linked by a covalent bond via a peptide linker therebetween, or are directly linked at the C-terminal, thereby forming a dimer-like structure, like the two-chain Fv. The antigen-binding fragment may be prepared using a protein hydrolytic enzyme (for example, Fab may be obtained by restricted truncation of the complete antibody with papain, and F(ab′)2 fragment may be obtained by restricted truncation of the complete antibody with pepsin), or may be prepared through genetic recombination techniques, but is not limited thereto.

### 2. Method of Screening Anticancer Drug

Still another aspect of the present disclosure provides a method of screening an anticancer drug, the method including: treating a cell expressing tryptophanyl-tRNA synthetase (WRS) with a candidate substance; measuring an expression level or activity level of the WRS in the cell treated with the candidate substance; and determining the candidate substance as an anticancer drug when the expression level or activity level of the WRS decreases compared to a control group not treated with the candidate substance.

The overlapping descriptions are the same as those described in “1. Method of Ameliorating or Treating Cancer” and are therefore omitted.

In the present disclosure, the candidate substance may be a known substance or a novel substance, and may be at least one selected from the group consisting of a peptide, a protein, a non-peptide compound, a synthetic compound, a fermentation product, a cell extract, a plant extract, and an animal tissue extract, but is not limited thereto.

In addition, in the present disclosure, the screening method may be performed in vivo or in vitro, and is not particularly limited.

In the present disclosure, the measuring f the expression level of the WRS may be performed by measuring an mRNA expression level of WRS. The measurement of the mRNA expression level is a process of confirming the presence and expression level of mRNA of the WRS in order to screen an anticancer drug, and may be performed by measuring the amount of mRNA. As a formulation for measuring the mRNA expression level, a primer pair, a probe, or an antisense nucleotide for the mRNA of WRS may be used, and the mRNA expression level may be measured by one or more methods selected from the group consisting of RT-PCR, competitive RT-PCR, real-time RT-PCR, RNase protection assay (RPA), Northern blotting, and DNA chips, but is not limited thereto.

In addition, in the present disclosure, the measuring of the activity level of the WRS may be performed by measuring an expression level of a WRS protein. The measurement of the protein expression level is a process of confirming the presence and expression level of a protein expressed in a sample collected and separated from a cell line or a patient in order to screen an anticancer drug, and may be performed by measuring the amount of protein using an antibody that specifically binds to the protein of the gene. An antibody may be used as a formulation for measuring the protein expression level, and the protein expression level may be measured by one or more methods selected from the group consisting of Western blotting, enzyme linked immunosorbent assay (ELISA), radioimmunoassay (RIA), radioimmunodiffusion, Ouchterlony immunodiffusion, rocket immunoelectrophoresis, tissue immunostaining, dual luciferase reporter assay, immunoprecipitation assay, complement fixation assay, fluorescence activated cell sorter (FACS), and protein chips, but is not limited thereto.

In the screening method of the present disclosure, the expression level of the WRS gene or the expression level of the WRS protein in a cell is measured in the absence of an anticancer drug candidate substance, the expression level of the WRS or the expression level of the WRS protein is also measured in the presence of the candidate substance, and the two expression levels are compared, and thereafter, when the expression level of the WRS gene or the expression level of the WRS protein in the presence of the candidate substance is reduced compared to the expression level in the absence of the candidate substance, the candidate substance is determined as an anticancer drug, such that the screening method may be usefully utilized in screening an anticancer drug.

In the present disclosure, when the expression level or activity level of the WRS is reduced by 18 or more, 5% or more, 10% or more, 15% or more, 20% or more, 258 or more, 30% or more, 35% or more, 40% or more, 45% or more, or 50% or more compared to a control group not treated with the candidate substance, the substance may be significantly determined as an candidate anticancer drug.

Hereinafter, the present disclosure will be described in detail with reference to Examples.

However, the following Examples illustrate only the present disclosure in detail, and the present disclosure is not limited by the following Examples.

### Example 1

Tryptophanyl-tRNA synthetase (WRS), which is an essential enzyme that binds tryptophan to tRNA through aminoacylation during a protein translation process, is secreted from immune cells in response to bacterial or viral infection, and full-length WRS is known to be spliced or truncated into mini-WRS, T1-WRS, or T2-WRS fragments. The following experiments were performed to determine whether the WRS is expressed by cancer cells.

First, in order to determine whether cancer cells induce WRS expression in human monocytes, a normal cell line or a cancer cell line was co-cultured with human monocytes, and then an expression level of WRS was measured.

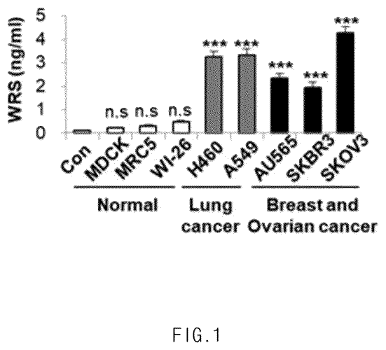

As a result, when human monocytes were co-cultured with a normal cell line (MDCK, MRC5, or WI-26), no significant change in the WRS expression level was observed. However, when human monocytes were co-cultured with lung cancer cell line (H460 or A549), breast cancer cell line (AU565 or SKBR3), or ovarian cancer cell line (SKOV3), a significant increase in the WRS expression level was observed (FIG. 1).

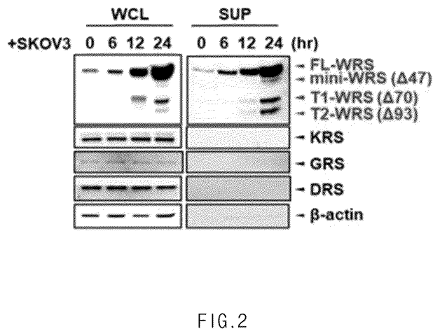

In addition, as a result of co-culturing human monocytes with ovarian cancer cell line SKOV3 and then confirming WRS expression in each of a whole cell lysate (WCL) and a supernatant (SUP), it was confirmed that WRS and fragments thereof were expressed in both the whole cell lysate and the supernatant (FIG. 2).

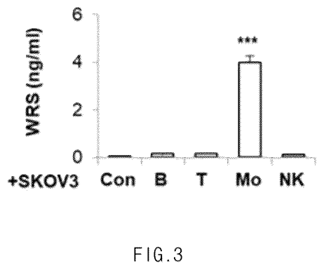

Next, as a result of co-culturing human immune cells (B cells, T cells, monocytes, and NK cells) with ovarian cancer cell line SKOV3 to confirm which immune cells express WRS, it was confirmed that WRS was expressed specifically in monocytes (FIG. 3).

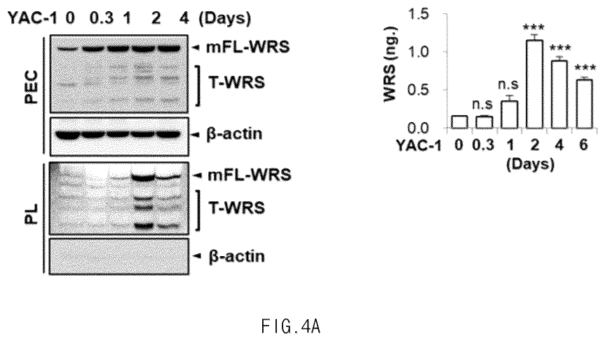

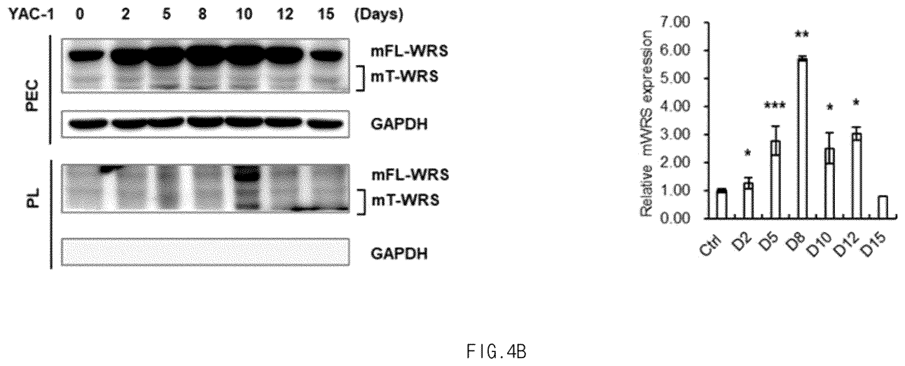

Finally, as a result of performing intraperitoneal injection of cancer cell line YAC-1 into a mouse and then measuring WRS expression in a peritoneal exudate cell (PEC) and a peritoneal lavage (PL) using Western blotting, ELISA, and RT-qPCR, it was confirmed that long-term expression of WRS and fragments thereof was shown in both the peritoneal exudate cell and the peritoneal lavage (FIGS. 4A and 4B).

Through the above experiments, it was confirmed that the expression of WRS and fragments thereof were induced by cancer cells, and the expression was monocyte-specific.

### Example 2

### Confirmation of MDSC Differentiation and Cancer Cell Proliferation Promotion Effects of WRS

The myeloid-derived suppressor cells (MDSCs) are known to be induced to differentiate by cancer and inhibit anticancer immunity of immune cells. In order to confirm the function of the WRS in the process of inducing differentiation of MDSCs by cancer cells, the following experiments were performed.

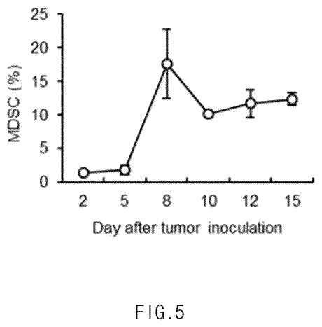

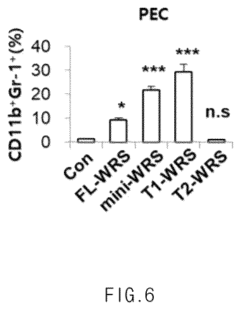

First, it was confirmed that differentiation of MDSCs was induced when the cancer cell line YAC-1 was intraperitoneally injected into the mouse (FIG. 5), and even when the WRS or fragments thereof were intraperitoneally injected into the mouse, MDSC differentiation was significantly promoted by mini-WRS and T1-WRS among the WRS fragments (FIG. 6).

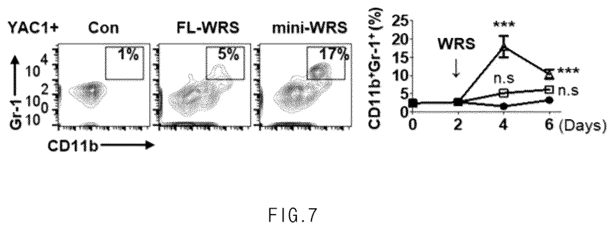

In addition, as a result of intraperitoneally injecting cancer cell line YAC-1 and full-length WRS or mini-WRS together into a mouse and then measuring a degree of MDSC differentiation by flow cytometry, it was confirmed that MDSC differentiation was significantly promoted when treated with mini-WRS (FIG. 7).

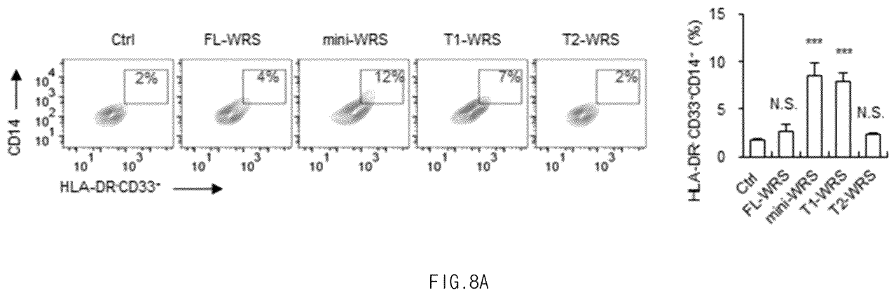

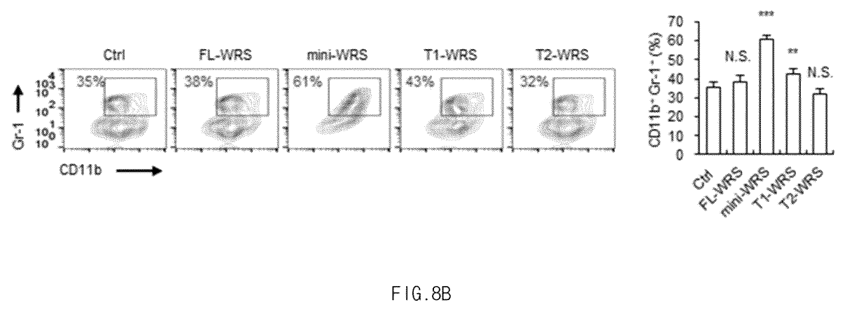

Next, as a result of measuring a level of MDSC differentiation after treating human umbilical cord blood CD34⁺ hematopoietic stem cells (HSCs) with hGM-CSF to differentiate the HSCs into MDSCs, and then performing treatment with the WRS or fragments thereof, it was confirmed that MDSC differentiation was significantly increased by mini-WRS and T1-WRS (FIG. 8A). In addition, even when mouse bone marrow hematopoietic stem cells were treated with mGM-CSF to differentiate the stem cells into MDSCs, and then treatment with the WRS or fragments thereof was performed, it was confirmed that MDSC differentiation was significantly increased by mini-WRS and T1-WRS, similar to the human hematopoietic stem cells (FIG. 8B).

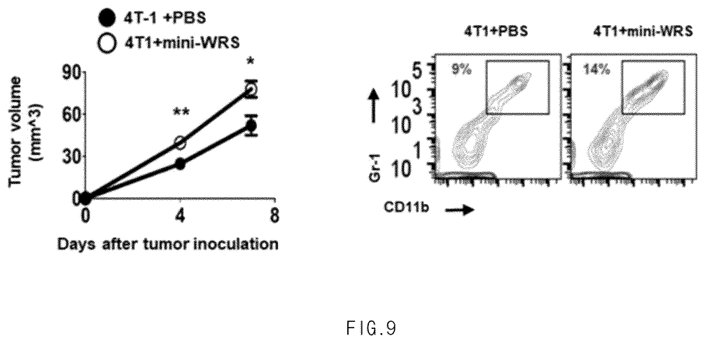

Finally, when mini-WRS was injected into a cancer nodule after forming a solid cancer by implanting cancer cell line 4T1 subcutaneously in a mouse, it was confirmed that the differentiation of MDSCs was promoted and the tumor volume also significantly increased (FIG. 9).

Through the above experiments, it was confirmed that the differentiation of MDSCs that inhibit anticancer immunity of immune cells was promoted by the WRS, in particular, the differentiation of MDSCs was significantly promoted by mini-WRS and T1-WRS fragments, and the proliferation of cancer cells was also promoted by mini-WRS.

### Example 3

### Confirmation of T Cell Differentiation Inhibitory Effect of WRS

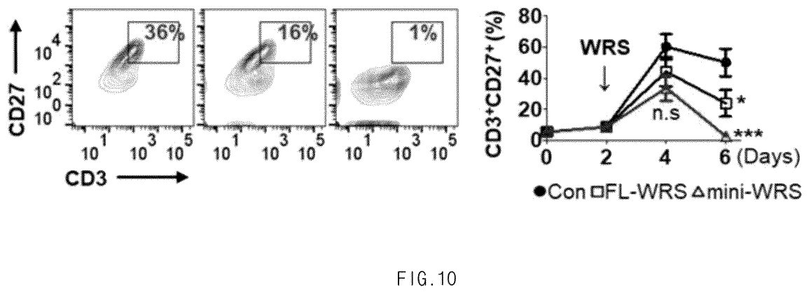

In order to determine the effect of the WRS on T cell differentiation, first, cancer cell line YAC-1 and full-length WRS or mini-WRS were intraperitoneally injected together into a mouse, and then, a degree of T cell differentiation was measured by flow cytometry. As a result, it was confirmed that T cell differentiation was inhibited by full-length WRS treatment and T cell differentiation was significantly inhibited by mini-WRS treatment (FIG. 10).

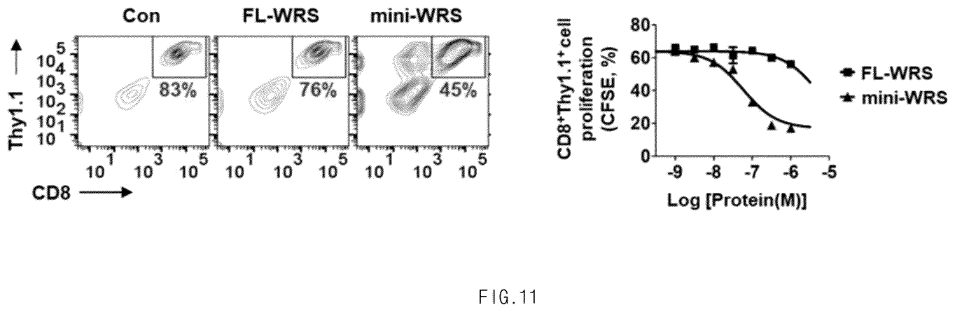

Additionally, an FBL cell line expressing gag as an antigen and a splenocyte from a TCR-gag transgenic mouse were co-cultured to proliferate antigen-specific T cells, treatment with full-length WRS or mini-WRS was performed, and then, a level of T cell differentiation and proliferation was measured. As a result, it was confirmed that T cell differentiation and proliferation were inhibited by the full-length WRS treatment and T cell differentiation and proliferation were significantly inhibited by the mini-WRS treatment (FIG. 11).

Through the above experiments, it was confirmed that the differentiation of T cells was inhibited by the WRS, and in particular, the differentiation and proliferation of T cells were significantly inhibited by the mini-WRS fragments.

### Example 4

### Analysis of Binding Affinity of WRS to LILRB Receptor

It is known that the differentiation of MDSCs is associated with an LILRB2 receptor. Therefore, in order to confirm whether the WRS promotes the differentiation of MDSCs by acting on the LILRB receptor, the binding affinity of the WRS and the fragments thereof to the LILRB receptor was measured using SPR analysis.

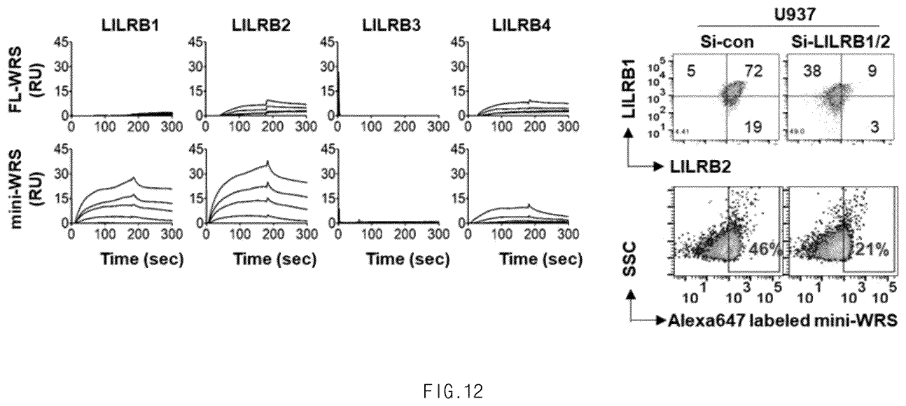

As a result, the binding affinity of mini-WRS to LILRB1 and LILRB2 was measured to be higher than that of full-length WRS, and when the expression of LILRB1 and LILRB2 was suppressed in monocytic cell line U937, it was confirmed that the binding affinity of mini-WRS to cells was significantly reduced (FIG. 12).

Through the above experiments, it was confirmed that the differentiation of MDSCs was regulated by binding of the WRS to the LILRB receptor.

### Example 5

### Confirmation of Cancer Growth Inhibitory Effect of WRS Target Antibody

Through the experiments performed in Examples 1 to 4, it was confirmed that the differentiation of MDSCs was promoted by the WRS, while the differentiation and proliferation of T cells were inhibited by the WRS, such that the proliferation of cancer cells and tumor volume were increased. Accordingly, after preparing a WRS target antibody capable of inhibiting the activity of WRS, the following experiments were performed to confirm the cancer growth inhibitory effect of the antibody.

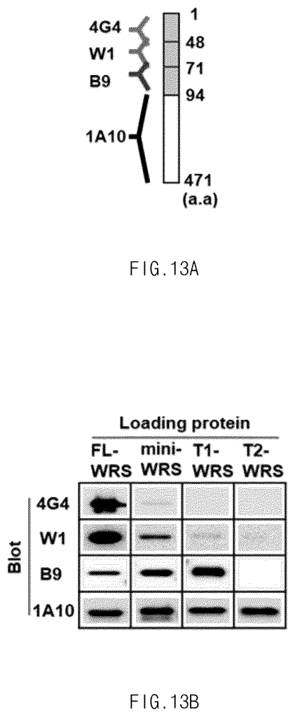

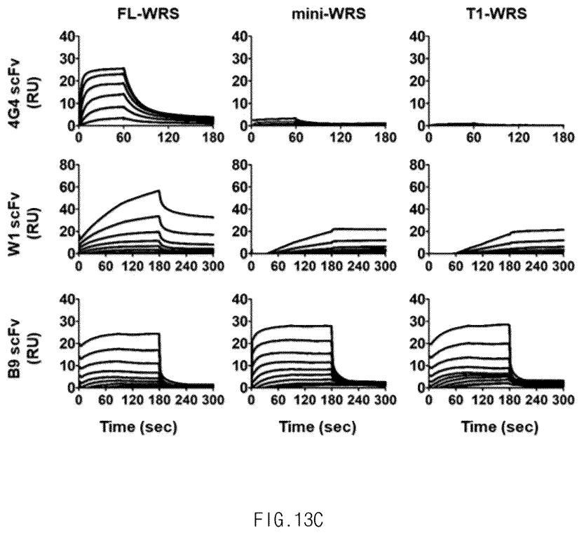

First, as antibodies to the WRS and the fragments thereof, 4G4, W1, B9, and 1A10 were prepared (FIG. 13A), and their binding affinity to the WRS and the fragments thereof was measured using Western blotting and SPR analysis (FIGS. 13B and 13C). The binding affinity of the 4G4 antibody was high only to the full-length WRS, and the binding affinity of the W1 antibody was high to the full-length WRS and mini-WRS. The binding affinity of the B9 antibody was high to the full-length WRS, mini-WRS, and T1-WRS, and the binding affinity of the 1A10 antibody was high to all of the full-length WRS, mini-WRS, T1-WRS, and T2-WRS.

Thereafter, in order to confirm whether the antibodies have a cancer growth inhibitory effect, the cancer cell line 4T1 was implanted subcutaneously in a mouse to form a solid cancer, the WRS antibody was injected into a cancer nodule, and then a tumor volume was measured.

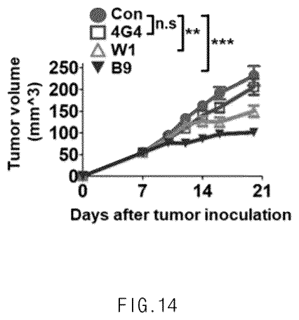

As a result, it was confirmed that the 4G4 antibody did not significantly reduce the tumor volume compared to the control group, but the W1 antibody and the B9 antibody reduced a tumor volume, and the B9 antibody significantly reduced a tumor volume (FIG. 14).

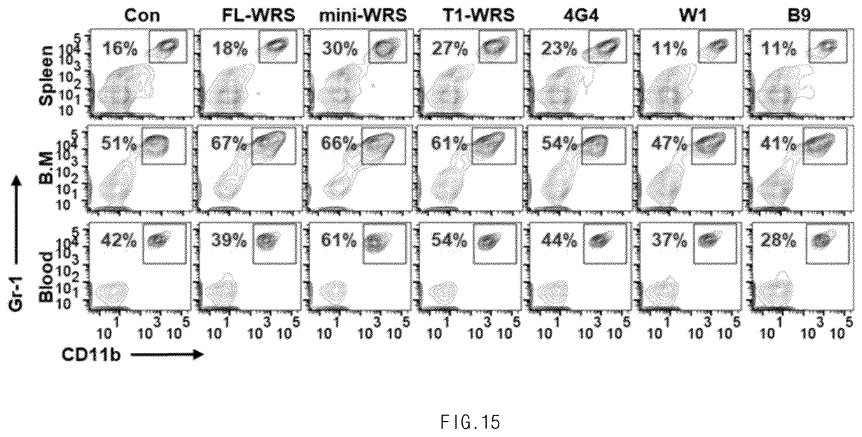

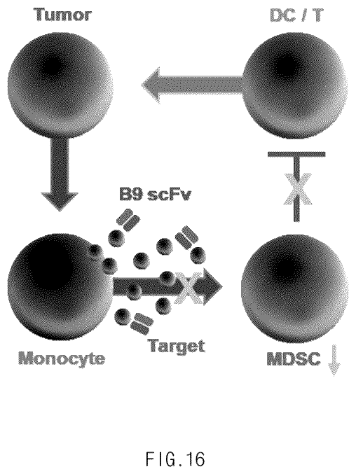

In addition, in the case of the treatment with the mini-WRS or T1-WRS, the degree of MDSC differentiation in the spleen, bone marrow, and blood significantly increased, but in the case of the treatment with the W1 antibody or the B9 antibody, the degree of MDSC differentiation decreased, and the B9 antibody significantly reduced the degree of MDSC differentiation (FIG. 15).

Through the above experiments, it was confirmed that the cancer growth and the MDSC differentiation were inhibited by the WRS target antibody, and in particular, the cancer growth and the MDSC differentiation were significantly inhibited by the B9 antibody. Therefore, it is expected that the WRS target antibody may be usefully utilized as an anticancer drug.

As set forth above, in the method of ameliorating or treating cancer, the tryptophanyl-tRNA synthetase (WRS) to be targeted is secreted by cancer cells and induces and promotes the differentiation of myeloid-derived suppressor cells (MDSCs), thereby having the activity of inhibiting anticancer immunity of immune cells. Therefore, in the case of the present disclosure using the WRS inhibitor as an active ingredient, the expression or activity of the WRS is inhibited and the differentiation of MDSCs is inhibited, such that the proliferation of cancer cells may be effectively inhibited. Therefore, the present disclosure may be used as an effective method of ameliorating or treating cancer and a method of enhancing or promoting anticancer efficacy of an anticancer drug, and further, may be usefully utilized as a method of screening a substance that inhibits the expression or activity of WRS as an anticancer drug.

Hereinabove, although the present disclosure has been described with reference to exemplary embodiments and the accompanying drawings, the present disclosure is not limited thereto, but may be variously modified and altered by those skilled in the art to which the present disclosure pertains without departing from the spirit and scope of the present disclosure claimed in the following claims.

What is claimed is:

1. A method of ameliorating or treating cancer, the method comprising administering a tryptophanyl-tRNA synthetase (WRS) inhibitor to a subject in need thereof.

2. The method of claim 1, wherein the WRS inhibitor is one or more selected from the group consisting of an antisense oligonucleotide, siRNA, shRNA, and miRNA that complementarily bind to a WRS gene.

3. The method of claim 1, wherein the WRS inhibitor is one or more selected from the group consisting of a small molecule compound, an aptamer, and an antibody that specifically bind to a WRS protein.

4. The method of claim 3, wherein the antibody contains a heavy chain variable region containing an amino acid sequence of SEQ ID NO: 1 and a light chain variable region containing an amino acid sequence of SEQ ID NO: 2.

5. The method of claim 1, wherein the WRS inhibitor inhibits differentiation of myeloid-derived suppressor cells (MDSCs).

6. The method of claim 1, wherein the WRS is mini-WRS or T1-WRS.

7. The method of claim 1, wherein the cancer is one or more selected from the group consisting of stomach cancer, colon cancer, pancreatic cancer, liver cancer, cervical cancer, breast cancer, ovarian cancer, head and neck cancer, leukemia, carcinoid, prostate cancer, lung cancer, bladder cancer, endometrial cancer, melanoma, kidney cancer, testicular cancer, glioma, thyroid cancer, skin cancer, and lymphoma.

8. The method of claim 1, wherein the cancer is one or more selected from the group consisting of lung cancer, breast cancer, and ovarian cancer.

9. A method of enhancing or promoting anticancer efficacy of an anticancer drug, the method comprising administering the WRS inhibitor of claim 1 to a subject in need thereof.

10. The method of claim 9, wherein the WRS is mini-WRS or T1-WRS.

11. The method of claim 9, wherein the anticancer drug is a cancer immunotherapy drug.

12. A method of screening an anticancer drug, the method comprising:
treating a cell expressing tryptophanyl-tRNA synthetase (WRS) with a candidate substance;
measuring an expression level or activity level of the WRS in the cell treated with the candidate substance; and
determining the candidate substance as an anticancer drug when the expression level or activity level of the WRS decreases compared to a control group not treated with the candidate substance.

13. The method of claim 12, wherein the WRS is mini-WRS or T1-WRS.

14. An antibody that specifically binds to tryptophanyl-tRNA synthetase (WRS) or an antigen-binding fragment thereof, the antibody or the antigen-binding fragment thereof comprising a heavy chain variable region containing an amino acid sequence of SEQ ID NO: 1; and
a light chain variable region containing an amino acid sequence of SEQ ID NO: 2.

15. The antibody or the antigen-binding fragment thereof of claim 14, wherein the WRS is mini-WRS or T1-WRS.
